(19) United States
(12) Patent Application Publication
(10) Pub. No.: US 2024/0374855 A1
(43) Pub. Date: Nov. 14, 2024

(54) Color-Changing Oxygen Tubing Device

(71) Applicant: Curt Ekey, Weirton, WV (US)

(72) Inventor: Curt Ekey, Weirton, WV (US)

(21) Appl. No.: 18/661,779

(22) Filed: May 13, 2024

Related U.S. Application Data
(60) Provisional application No. 63/466,021, filed on May 12, 2023.

(51) Int. Cl. A61M 16/10

(52) U.S. Cl. CPC A61M 16/1005; A61M 2016/1025; A61M 2205/584

(57) ABSTRACT

A color-changing oxygen tubing device is provided. The device is comprised of an oxygen tubing that changes color when the presence of oxygen (or a specific saturation of oxygen) flows through the tubing. The tubing may change from transparent to a first color or from a first color to a second color.

### CROSS-REFERENCE TO RELATED APPLICATION

The present application claims priority to, and the benefit of, U.S. Provisional Application No. 63/466,021, which was filed on May 12, 2023, and is incorporated herein by reference in its entirety.

### FIELD OF THE INVENTION

The present invention relates generally to the field of medical devices. More specifically, the present invention relates to a color-changing oxygen tubing device that changes color when the presence of oxygen (or a specific saturation of oxygen) flows through the tubing. Accordingly, the present disclosure makes specific reference thereto. Nonetheless, it is to be appreciated that aspects of the present invention are also equally applicable to other like applications, devices, and methods of manufacture.

### BACKGROUND

Individuals with respiratory conditions often rely on supplemental oxygen to maintain optimal breathing functionality. This is particularly true for those suffering from Chronic Obstructive Pulmonary Disease (COPD), who require a meticulously calibrated amount of oxygen to manage their condition effectively. However, one of the challenges faced in such treatments is the difficulty in accurately determining the precise amount/saturation of oxygen being delivered through the oxygen tubing to the individual. This issue is compounded by the fact that any deviation from the required oxygen levels can lead to serious health risks. Maintaining the right oxygen saturation is critical, as it ensures that the patient's blood carries enough oxygen to meet their body's needs. Incorrect oxygen levels can be incredibly dangerous, especially for those who depend on this external support for their respiratory needs. Thus, it becomes imperative to have reliable methods and tools to monitor and adjust the oxygen flow accurately, ensuring that patients receive the right amount of oxygen at all times.

Therefore, there exists a long-felt need in the art for oxygen monitoring. There also exists a long-felt need in the art for a color-changing oxygen tubing device. Further, there exists a long-felt need in the art for a color-changing oxygen tubing device that allows an individual to visually confirm proper oxygen levels are being delivered to a patient via oxygen tubing.

The subject matter disclosed and claimed herein, in one embodiment thereof, comprises a color-changing oxygen tubing device. The device is comprised of an oxygen tubing that changes color when the presence of oxygen (or a specific saturation of oxygen) flows through the tubing. The tubing may change from transparent to a first color or from a first color to a second color.

In this manner, the color-changing oxygen tubing device of the present invention accomplishes all the foregoing objectives and provides a device for oxygen monitoring. More specifically, the device that allows an individual to visually confirm proper oxygen levels are being delivered to a patient via oxygen tubing via the color changing of the tubing.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a color-changing oxygen tubing device comprised of a medical-grade tubing that changes color when oxygen is passed through the tubing. The tubing may be made from any medical-grade tubing material known in the art. The tubing is comprised of a first end comprised of a first opening and a second end comprised of a second opening.

In one embodiment, the tubing is transparent and changes to a first color when oxygen travels through the tubing. In a second embodiment, the tubing is a first color and changes to a second color when oxygen travels through the tubing. In either embodiment, the tubing composition and/or an interior surface of the tubing may include the presence of (i.e., infused, coated, etc.) a secondary compound/material that allows the tubing to change color. The compound is preferably within the interior space of the tubing.

In one embodiment, the compound is comprised of an oxygen-indicating dye that changes color in response to a reduction-oxidation reaction within the interior space. In another embodiment, the compound is comprised of an oxygen absorber and indicator designed to absorb oxygen and indicate the presence of oxygen simultaneously. In a further embodiment, the compound is comprised of a photoluminescent pigment that may glow in the absence of oxygen in the interior space but stop glowing when oxygen is present in the interior space (or vice versa). Another embodiment may be comprised of an electronic polymer that may change electrical properties in the presence of oxygen within the interior space to cause a color change. In one embodiment, the compound is comprised of an inorganic compound that changes color in response to oxygen within the interior space. Furthermore, the compound may only react to a specific level of oxygen saturation within the interior space.

The present invention is also comprised of a method of using the device. First, a device is provided comprised of a tubing comprised of a compound that changes color (and/or the color of the tubing) when oxygen (or a specific saturation of oxygen) is flowing through the tubing. Then, a first end of the tubing can be attached to an oxygen flowmeter and/or any oxygen monitoring/supplying device. Then, a cannula can be inserted into the second end of the tubing. In one embodiment, the cannula may be fixedly attached to the second end during the manufacturing process. Then, the cannula can be inserted into at least one nostril (or other body area) of the user. Finally, a secondary user can observe the tubing to ensure that the tubing changes from transparent to a first color or from a first color to a second color to confirm oxygen and/or the proper saturation of oxygen is flowing through the tubing to the patient.

Accordingly, the color-changing oxygen tubing device of the present invention is particularly advantageous as it provides a device for oxygen monitoring. More specifically, the device that allows an individual to visually confirm proper oxygen levels are being delivered to a patient via oxygen tubing via the color changing of the tubing. In this manner, the color-changing oxygen tubing device overcomes the limitations of existing oxygen delivery methods and devices known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a color-changing oxygen tubing device of the present invention in accordance with the disclosed architecture;

FIG. 2 illustrates an enhanced perspective view of a first end of one potential embodiment of a color-changing oxygen tubing device of the present invention in accordance with the disclosed architecture;

FIG. 3 illustrates an enhanced perspective view of a first end of one potential embodiment of a color-changing oxygen tubing device of the present invention in accordance with the disclosed architecture; and

FIG. 4 illustrates a flowchart of a method of using one potential embodiment of a color-changing oxygen tubing device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for oxygen monitoring. There also exists a long-felt need in the art for a color-changing oxygen tubing device. Further, there exists a long-felt need in the art for a color-changing oxygen tubing device that allows an individual to visually confirm proper oxygen levels are being delivered to a patient via oxygen tubing.

The present invention, in one exemplary embodiment, is comprised of a color-changing oxygen tubing device. The device is comprised of medical-grade tubing that changes color when oxygen is passed through the tubing. The tubing may be made from any medical-grade tubing material known in the art and is comprised of a first end comprised of a first opening and a second end comprised of a second opening.

In one embodiment, the tubing is transparent and changes to a first color when oxygen travels through the tubing. In a second embodiment, the tubing is a first color and changes to a second color when oxygen travels through the tubing. In cither embodiment, the tubing composition and/or an interior surface of the tubing may include the presence of (i.e., infused, coated, etc.,) a secondary compound/material that allows the tubing to change color. The compound is preferably within the interior space of the tubing.

In one embodiment, the compound is comprised of an oxygen-indicating dye that changes color in response to a reduction-oxidation reaction within the interior space. In another embodiment, the compound is comprised of an oxygen absorber and indicator designed to absorb oxygen and indicate the presence of oxygen simultaneously. In a further embodiment, the compound is comprised of a photoluminescent pigment that may glow in the absence of oxygen in the interior space but stop glowing when oxygen is present in the interior space (or vice versa). Another embodiment may be comprised of an electronic polymer that may change electrical properties in the presence of oxygen within the interior space to cause a color change. In one embodiment, the compound is comprised of an inorganic compound that changes color in response to oxygen within the interior space. Furthermore, the compound may only react to a specific level of oxygen saturation within the interior space.

The present invention is also comprised of a method of using the device. First, a device is provided comprised of a tubing comprised of a compound that changes color (and/or the color of the tubing) when oxygen (or a specific saturation of oxygen) is flowing through the tubing. Then, a first end of the tubing can be attached to an oxygen flowmeter and/or any oxygen monitoring/supplying device. Then, a cannula can be inserted into the second end of the tubing. In one embodiment, the cannula may be fixedly attached to the second end during the manufacturing process. Then, the cannula can be inserted into at least one nostril (or other body area) of the user. Finally, a secondary user can observe the tubing to ensure that the tubing changes from transparent to a first color or from a first color to a second color to confirm oxygen and/or the proper saturation of oxygen is flowing through the tubing to the patient.

Accordingly, the color-changing oxygen tubing device of the present invention is particularly advantageous as it provides a device for oxygen monitoring. More specifically, the device that allows an individual to visually confirm proper oxygen levels are being delivered to a patient via oxygen tubing via the color changing of the tubing. In this manner, the color-changing oxygen tubing device overcomes the limitations of existing oxygen delivery methods and devices known in the art.

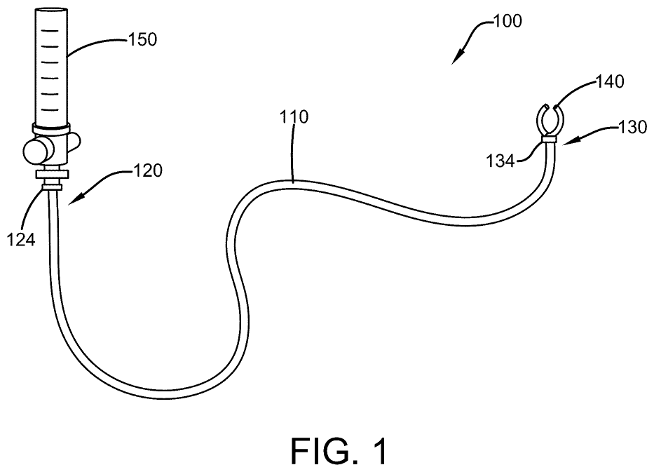

Referring initially to the drawings, FIG. 1 illustrates a perspective view of one potential embodiment of a color-changing oxygen tubing device 100 of the present invention in accordance with the disclosed architecture. The device 100 is comprised of medical-grade tubing that changes color when oxygen is passed through the tubing 110. The device 100 is comprised of a tubing 110. The tubing 110 may be made from any medical-grade tubing material known in the art. This includes, but is not limited to, silicone tubing, PVC tubing, PTFE tubing, polyurethane tubing, polyethylene tubing, TPE tubing, latex rubber tubing, etc. The tubing 110 may be any length and diameter.

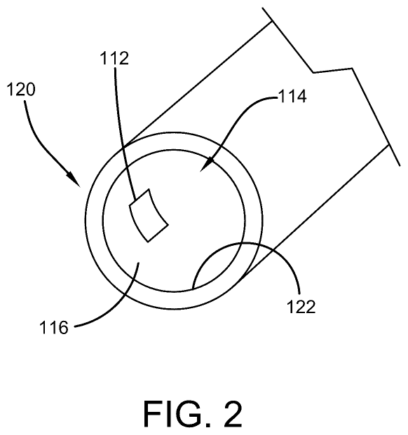

The tubing 110 is comprised of a first end 120 comprised of a first opening 122, as seen in FIG. 2. The end 120 may be comprised of at least one attachment member 124. The member 124 may be any structure/connector that allows the first end 120 to attach to an oxygen flowmeter 150 or other similar oxygen delivery/monitoring device. In one embodiment, the first end 120 is tapered to facilitate attachment to the flowmeter 150.

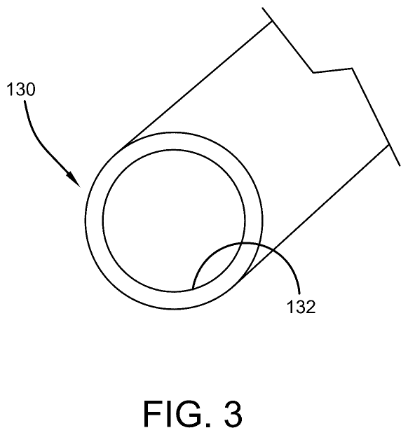

The tubing 110 is comprised of a second end 130 comprised of a second opening 132, as seen in FIG. 3. The end 130 may be comprised of at least one attachment member 134. The member 134 may be any structure/connector that allows the second end 130 to attach to a nasal cannula 140. In one embodiment, the second end 130 is tapered to facilitate attachment to the cannula 140. In a different embodiment, the cannula 140 is fixedly attached to the tubing 110. The cannula 140 may be any cannula style known in the art.

In one embodiment, the tubing 110 is transparent and changes to a first color when oxygen travels through the tubing 110. In a second embodiment, the tubing 110 is a first color and changes to a second color when oxygen travels through the tubing 110. The color(s) of the tubing 110 may be any color. In either embodiment, the tubing 110 composition and/or an interior surface 116 of the tubing 110 may include the presence of (i.e., infused, coated, etc.,) a secondary compound/material 112 that allows the tubing 110 to change color. The compound 112 is preferably within the interior space 114 of the tubing 110.

In one embodiment, the compound 112 is comprised of an oxygen-indicating dye. The dye is preferably comprised of an organic compound that changes color in response to a reduction-oxidation reaction within the interior space 114.

In one embodiment, the compound 112 is comprised of an oxygen absorber and indicator designed to absorb oxygen and indicate the presence of oxygen simultaneously. In one embodiment, this may include iron-based oxygen absorbers combined with an oxygen-sensitive dye, wherein the dye changes color when the iron has reacted with oxygen in the interior space 114, indicating that the environment is no longer oxygen-free.

In one embodiment, the compound 112 is comprised of a photoluminescent pigment. The pigment may glow in the absence of oxygen in the interior space 114 but stop glowing when oxygen is present in the interior space 114 (or vice versa).

In one embodiment, the compound 112 is comprised of an electronic polymer. The polymer may change electrical properties in the presence of oxygen within the interior space 114 to cause a color change.

In one embodiment, the compound 112 is comprised of an inorganic compound that changes color in response to oxygen within the interior space 114.

In one embodiment, the compound 112 may only react to a specific level of oxygen saturation within the interior space 114.

The color change of the tubing 110 may include the entire length/surface area of the tubing 110 or only a specific area of the tubing 110.

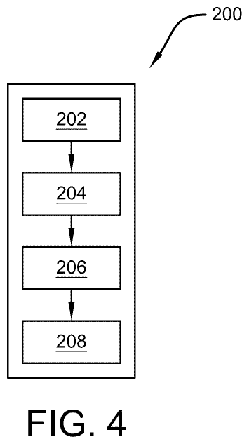

The present invention is also comprised of a method of using 200 the device 100, as seen in FIG. 4. First, a device 100 is provided comprised of a tubing 110 comprised of a compound 112 that changes color (and/or the color of the tubing 110) when oxygen (or a specific saturation of oxygen) is flowing through the tubing 110 [Step 202]. Then, a first end 120 of the tubing 110 can be attached to an oxygen flowmeter 150 and/or any oxygen monitoring/supplying device [Step 204]. Then, a cannula 140 can be inserted into the second end 130 of the tubing 110 [Step 206]. In one embodiment, the cannula 140 may be fixedly attached to the second end 130 during the manufacturing process. Then, the cannula 140 can be inserted into at least one nostril (or other body area) of the user [Step 208]. Finally, a secondary user can observe the tubing 110 to ensure that the tubing 110 changes from transparent to a first color or from a first color to a second color to confirm oxygen and/or the proper saturation of oxygen is flowing through the tubing 110 to the patient [Step 208].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “color-changing oxygen tubing device” and “device” are interchangeable and refer to the color-changing oxygen tubing device 100 of the present invention.

Notwithstanding the foregoing, the color-changing oxygen tubing device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the color-changing oxygen tubing device 100 as shown in the FIGS. are for illustrative purposes only, and that many other sizes and shapes of the color-changing oxygen tubing device 100 are well within the scope of the present disclosure. Although the dimensions of the color-changing oxygen tubing device 100 are important design parameters for user convenience, the color-changing oxygen tubing device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A color-changing oxygen tubing device comprising:
a tubing comprised of a first end comprised of a first opening and a second opening; and
a compound that changes color when exposed to oxygen.

2. The color-changing oxygen tubing device of claim 1, wherein the tubing is comprised of a silicone tubing, a PVC tubing, a PTFE tubing, a polyurethane tubing, a polyethylene tubing, a TPE tubing, or a latex rubber tubing.

3. The color-changing oxygen tubing device of claim 1, wherein the second end is comprised of a cannula.

4. The color-changing oxygen tubing device of claim 1, wherein the tubing is comprised of a transparent tubing.

5. The color-changing oxygen tubing device of claim 1, wherein the compound is infused or coated on an interior surface of the tubing.

6. The color-changing oxygen tubing device of claim 1, wherein the compound is comprised of an oxygen-indicating dye.

7. The color-changing oxygen tubing device of claim 1, wherein the compound is comprised of an oxygen absorber and indicator.

8. The color-changing oxygen tubing device of claim 1, wherein the compound is comprised of a photoluminescent pigment.

9. The color-changing oxygen tubing device of claim 1, wherein the compound is comprised of an electronic polymer.

10. The color-changing oxygen tubing device of claim 1, wherein the compound is comprised of an inorganic compound.

11. A color-changing oxygen tubing device comprising:
a tubing comprised of a first end comprised of a first opening and a second opening; and
a compound that changes from a first color to a second color when exposed to a specific saturation of oxygen.

12. The color-changing oxygen tubing device of claim 11, wherein the compound is infused or coated on an interior surface of the tubing.

13. The color-changing oxygen tubing device of claim 11, wherein the compound is comprised of an oxygen-indicating dye.

14. The color-changing oxygen tubing device of claim 11, wherein the compound is comprised of an oxygen absorber and indicator.

15. The color-changing oxygen tubing device of claim 11, wherein the compound is comprised of a photoluminescent pigment.

16. The color-changing oxygen tubing device of claim 11, wherein the compound is comprised of an electronic polymer.

17. The color-changing oxygen tubing device of claim 11, wherein the compound is comprised of an inorganic compound.

18. A method of using a color-changing oxygen tubing device, the method comprising the following steps:
providing a color-changing oxygen tubing device comprised of a tubing comprised of a compound that changes color when oxygen or a specific saturation of oxygen flows through the tubing;
attaching a first end of the tubing to an oxygen monitoring device or an oxygen supplying device;
inserting a cannula of a second end of the tubing into a body area of a user; and
observing the tubing changing color to confirm oxygen or a proper saturation of oxygen is flowing through the tubing.

19. The method of using a color-changing oxygen tubing device of claim 18, wherein the tubing changes from transparent to a first color due to the compound.

20. The method of using a color-changing oxygen tubing device of claim 18, wherein the tubing changes from a first color to a second color due to the compound.
